(19) United States
(12) Patent Application Publication
(10) Pub. No.: US 2014/0118688 A1
(43) Pub. Date: May 1, 2014

(54) OPHTHALMIC APPARATUS, IMAGING CONTROL APPARATUS, AND IMAGING CONTROL METHOD

(71) Applicant: Canon Kabushiki Kaisha, Tokyo (JP)

(72) Inventor: Takashi Masuda, Tokyo (JP)

(73) Assignee: Canon Kabushiki Kaisha, Tokyo (JP)

(21) Appl. No.: 14/050,530

(22) Filed: Oct. 10, 2013

(30) Foreign Application Priority Data
Nov. 1, 2012 (JP) 2012-242214

(51) Int. Cl. A61B 3/12; H04N 5/232

(52) U.S. Cl. CPC A61B 3/12; H04N 5/23212
USPC 351/206; 348/77

(57) ABSTRACT

An imaging control apparatus executes focusing operation for setting an in-focus state of an imaging optical system for an imaging device on an object by using the object image obtained by imaging the object illuminated by an illumination light source by using the imaging device. The imaging control apparatus changes an imaging setting to change the signal-to-noise ratio of the object image between the time of executing the focusing operation and other times. The imaging control apparatus processes the object image so as to maintain the tonality of the image displayed on a display device before and after an imaging setting is changed and displays the processed object image on the display device.

### BACKGROUND OF THE INVENTION

b 1. Field of the Invention

The present invention relates to an ophthalmic apparatus, an imaging control apparatus, and an imaging control method.

2. Description of the Related Art

In general, when using an ophthalmic apparatus typified by a non-mydriatic fundus camera, the operator performs upward, downward, leftward, rightward, forward, and backward positioning and focusing between the apparatus and the eye to be examined while seeing the fundus observation images captured by an imaging device. Recently, an ophthalmic apparatus has been known, which has an autofocus function for automatically focusing by using the fundus observation images captured by an imaging device.

The autofocus schemes of ophthalmic apparatuses can be roughly classified into two types. One type is the scheme of performing autofocusing by projecting split indices on the pupil of the eye to be examined and detecting the positional relationship between the captured index images by image processing as disclosed in Japanese Patent Laid-Open No. 5-95907. The autofocus scheme using index images is defined as an index image autofocus scheme. This index image autofocus scheme can accurately perform focusing on the pupil in the splitting direction on the pupil with respect to refractive errors such as the astigmatism of the eye to be examined but cannot accurately perform focusing on the indices in directions other than the splitting direction on the pupil.

The other type is the scheme of performing autofocusing by detecting the tone difference between fundus observation images by image processing without using the index images projected on the fundus of the eye to be examined when performing autofocusing as disclosed in Japanese Patent Laid-Open No. 2011-50532. The autofocus scheme using fundus images is defined as a fundus image autofocus scheme. This fundus image autofocus scheme can minimize an error caused by a refractive error such as the astigmatism of the optical system of the eye to be examined which is described in the index image autofocus scheme.

However, the fundus image autofocus scheme using the fundus observation images captured by an imaging device is susceptible to noise generated by the imaging device because the tone difference between fundus observation images as focusing targets is small. This leads to a degradation in focusing accuracy. The focusing accuracy is improved by increasing the signal-to-noise ratio (S/N ratio) between fundus observation images and noise generated by the imaging device. For example, it is possible to increase such a signal-to-noise ratio by increasing the illumination amount of an observation light source. It is, however, not always necessary to increase the signal-to-noise ratio (S/N ratio) between fundus observation images and noise from the imaging device during observation as in a case of positioning between the eye to be examined and the apparatus in the upward, downward, left, right, forward, and backward directions. For this reason, always increasing the illumination amount of the observation light source during observation will impose an unnecessarily heavy load on the operator.

### SUMMARY OF THE INVENTION

An embodiment of the present invention provides an ophthalmic apparatus which implements accurate focusing without imposing any unnecessarily heavy load on an object.

According to one aspect of the present invention, there is provided an imaging control apparatus comprising: a focusing unit configured to set an in-focus state of an imaging optical system on an object by using an object image obtained by imaging the object illuminated by a light source by using an imaging device; a change unit configured to change an imaging setting to change a signal-to-noise ratio of the object image obtained by the imaging at the time of executing focusing operation using the focusing unit and at other times; and a display control unit configured to process the object image so as to maintain tonality of the image displayed on a display unit before and after the change unit changes the imaging setting and display the processed object image on the display unit.

Also, according to another aspect of the present invention, there is provided an ophthalmic apparatus comprising: the imaging control apparatus described above; a light source; an imaging device; and an imaging optical system, wherein a fundus is imaged as an object.

Furthermore, according to another aspect of the present invention, there is provided an imaging control method comprising: a step of setting an in-focus state of an imaging optical system on an object by using an object image obtained by imaging an object illuminated by a light source by using an imaging device; a step of changing an imaging setting to change a signal-to-noise ratio of the object image obtained by the imaging at the time of executing focusing operation in the step of setting the in-focus state and at other times; and a step of processing the object image so as to maintain tonality of the image displayed on display unit before and after the imaging setting is changed in the step of changing and displaying the processed object image on the display unit.

Further features of the present invention will become apparent from the following description of exemplary embodiments with reference to the attached drawings.

### BRIEF DESCRIPTION OF THE DRAWINGS

FIG. 1 is a block diagram showing an example of the arrangement of a non-mydriatic fundus camera according to an embodiment;

FIG. 2 is a flowchart showing the operation of the non-mydriatic fundus camera according to the embodiment;

FIG. 3 is a view showing a fundus observation image and a focusing evaluation area for fundus image autofocusing;

FIG. 4 is a view showing a fundus observation image in a focusing evaluation area;

FIG. 5 is a graph showing the tone values of a fundus observation image without any influence of noise from an imaging device; and

FIG. 6 is a graph showing the tone values of a fundus observation image with noise from the imaging device being superimposed on the values.

### DESCRIPTION OF THE EMBODIMENTS

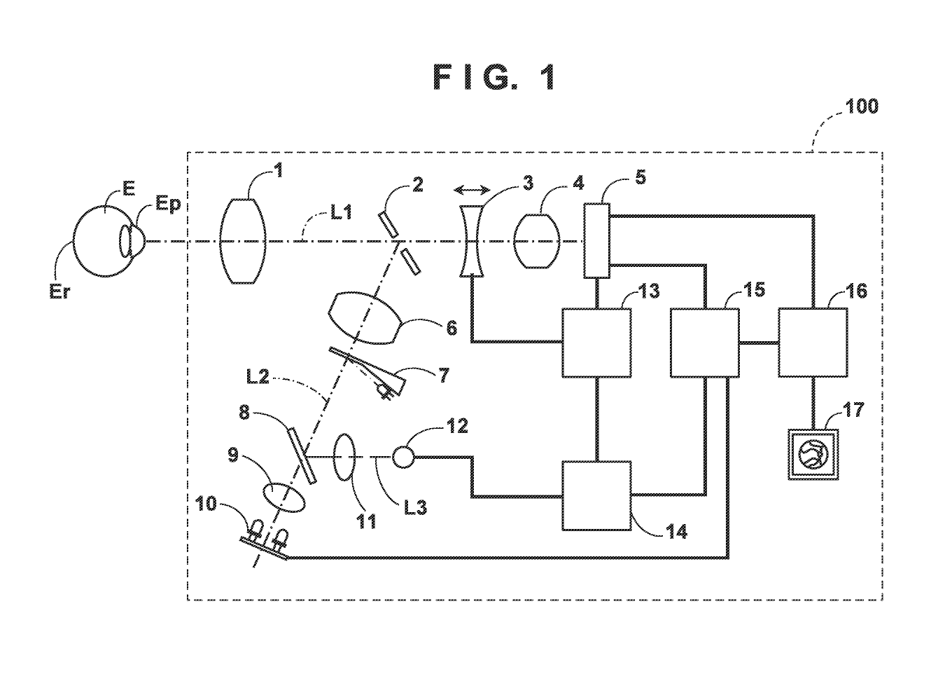

A preferred embodiment of the present invention will be described below with reference to the accompanying drawings. The embodiment of the present invention will be described below by exemplifying a case in which an imaging control apparatus of the present invention is applied to an ophthalmic apparatus, especially a non-mydriatic fundus camera. FIG. 1 is a block diagram showing the arrangement of a non-mydriatic fundus camera 100 according to the embodiment. The non-mydriatic fundus camera 100 of the embodiment has a function of performing fundus image autofocusing. The arrangement of the non-mydriatic fundus camera 100 will be described with reference to FIG. 1.

An objective lens 1, a perforated mirror 2, a focus lens 3, an imaging lens 4, and an imaging device 5 are sequentially arranged on an optical axis L1 extending to a fundus Er of an eye E to be examined to form an imaging optical system. The embodiment exemplifies an imaging optical system forming the non-mydriatic fundus camera. A fundus imaging optical system is formed for the eye E. On the other hand, a lens 6, an index projection unit 7, a dichroic mirror 8, a condenser lens 9, and an observation light source 10 are arranged on an optical axis L2 in the reflecting direction of the perforated mirror 2. In addition, a condenser lens 11 and an imaging light source 12 are arranged on an optical axis L3 in the reflecting direction of the dichroic mirror 8. The components on the optical axes L2 and L3 constitute an illumination optical system. The embodiment exemplifies a fundus illumination optical system which forms the non-mydriatic fundus camera 100.

The dichroic mirror 8 has the property of transmitting light in the wavelength range of the observation light source 10 and reflecting light in the wavelength range of the imaging light source 12. The observation light source 10 has a plurality of LEDs arranged to irradiate the eye to be examined with light having a wavelength in the infrared region. The imaging light source 12 is a light source which irradiates the fundus Er with light having a wavelength in the visible light region.

The non-mydriatic fundus camera 100 further includes a fundus image autofocus unit 13, a fundus camera control unit 14, an SN control unit 15, a display image processing unit 16, and a display unit 17. More specifically, the fundus image autofocus unit 13 is connected to the focus lens 3, the imaging device 5, and the fundus camera control unit 14, and calculates a focusing evaluation value from an image from the imaging device 5 and drives the focus lens 3 based on instructions from the fundus camera control unit 14. The SN control unit 15 is connected to the imaging device 5, the observation light source 10, the fundus camera control unit 14, and the display image processing unit 16, and sets the amplification factor of the imaging device 5 and the emitted light amount of the observation light source 10 based on instructions from the fundus camera control unit 14. The fundus camera control unit 14 is connected to the imaging light source 12, the fundus image autofocus unit 13, and the SN control unit 15, and performs light emission control for the imaging light source 12 and overall apparatus control which includes starting and stopping the operation of the fundus image autofocus unit 13 and SN control unit 15. The display image processing unit 16 is connected to the imaging device 5 and the display unit 17, and performs image processing for displaying images from the imaging device 5 on the display unit 17. The fundus image autofocus unit 13, the fundus camera control unit 14, the SN control unit 15, and the display image processing unit 16 described above constitute the imaging control unit of the non-mydriatic fundus camera 100.

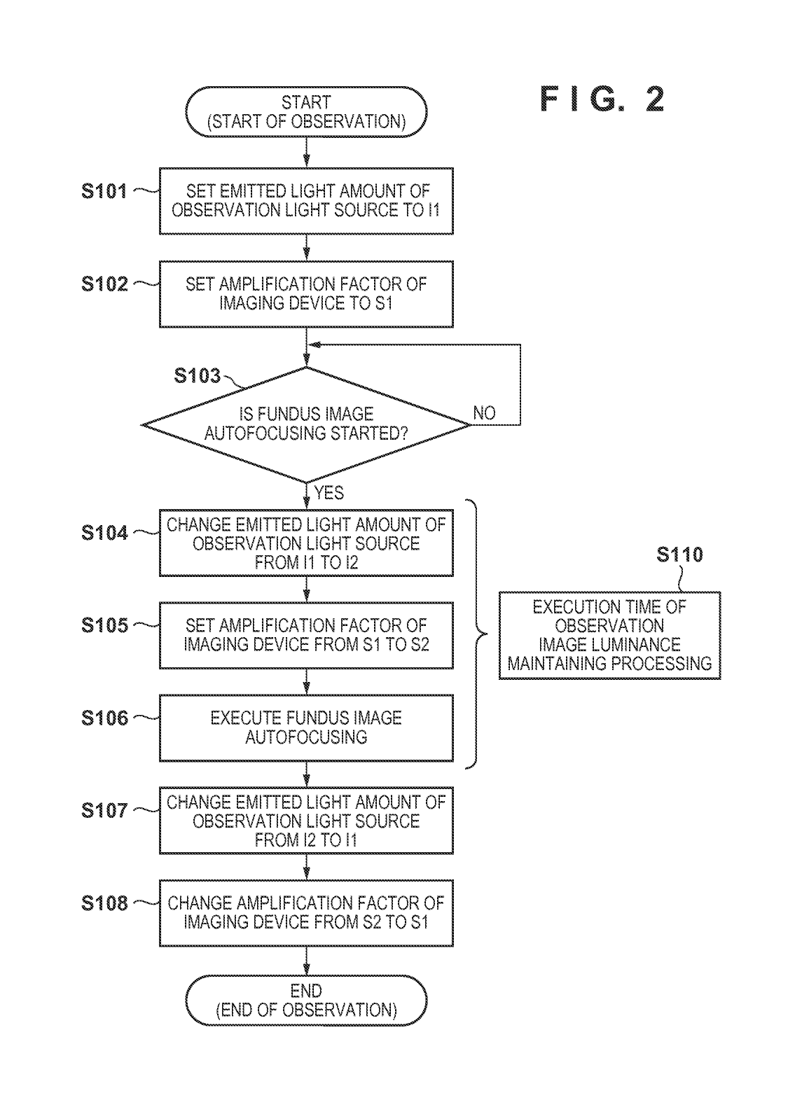

Operation starting from observation to imaging in the non-mydriatic fundus camera 100 according to this embodiment having the above arrangement will be described below. The observing operation will be described first with reference to the flowchart shown in FIG. 2. The flowchart of FIG. 2 shows the operation of the fundus image autofocus unit 13, fundus camera control unit 14, and SN control unit 15.

When the operator positions the eye E in front of the objective lens 1 and starts observation, the SN control unit 15 sets the emitted light amount of the observation light source 10 to I1 (step S101). When the observation light source 10 emits light at the emitted light amount I1 set by the SN control unit 15, the observation illumination light passes through a fundus illumination optical system extending from the observation light source 10 to the objective lens 1 and illuminates the fundus Er through a pupil Ep of the eye E. Reflected light from the fundus Er illuminated by the observation light source 10 reaches the imaging device 5 through a fundus imaging optical system including the objective lens 1, the perforated mirror 2, the focus lens 3, and the imaging lens 4.

At the same time of making setting for the observation light source 10, the SN control unit 15 sets the amplification factor of the imaging device 5 to S1 (step S102). With the set amplification factor S1, the imaging device 5 captures a fundus observation image. The display image processing unit 16 applies processing such as monochromatization and gamma curve computation to the fundus observation image. The display unit 17 displays the resultant image. The operator performs positioning between the eye E and the non-mydriatic fundus camera 100 in the upward, downward, leftward, rightward, forward, and backward directions by moving the non-mydriatic fundus camera 100 upward, downward, leftward, rightward, forward, and backward with a console (not shown) while seeing the fundus observation image displayed on the display unit 17.

When the operator performs operation to issue an instruction to start fundus image autofocusing or the apparatus determines by itself that it is possible to perform focusing from the fundus image (YES in step S103), the apparatus starts focusing. The fundus image autofocus unit 13 executes focusing operation to focus the imaging optical system on an object (the fundus in this embodiment) by using the object image obtained by causing the imaging device 5 to image the object illuminated by the observation light source 10 as a light source. The apparatus changes imaging settings so as to increase the signal-to-noise ratio of the object image acquired by imaging using the imaging device 5 during the execution of focusing operation as compared with other times, that is, during a focusing operation non-execution time.

In this embodiment, when changing the above imaging settings, the apparatus increases the emitted light amount of the observation light source 10 during execution of focusing operation and also decreases the amplification factor of a signal from the imaging device 5. First of all, the SN control unit 15 changes the emitted light amount of the observation light source 10 from I1 to I2. In this case, the emitted light amount I2 is larger than the emitted light amount I1 (I2>I1). The observation light source 10 emits light at the emitted light amount I2 set by the SN control unit 15 (step S104). At almost the same time of setting the emitted light amount I2, the SN control unit 15 changes the amplification factor of the imaging device 5 from S1 to S2. The amplification factor S2 is smaller than the amplification factor S1 (S1>S2) (step S105). Note that the apparatus performs the processing of maintaining the observation luminance constant in steps S104 and S105. This processing will be described later. Note that the apparatus performs the processing of maintaining the observation luminance constant during fundus image autofocusing (to be described below) (step S110).

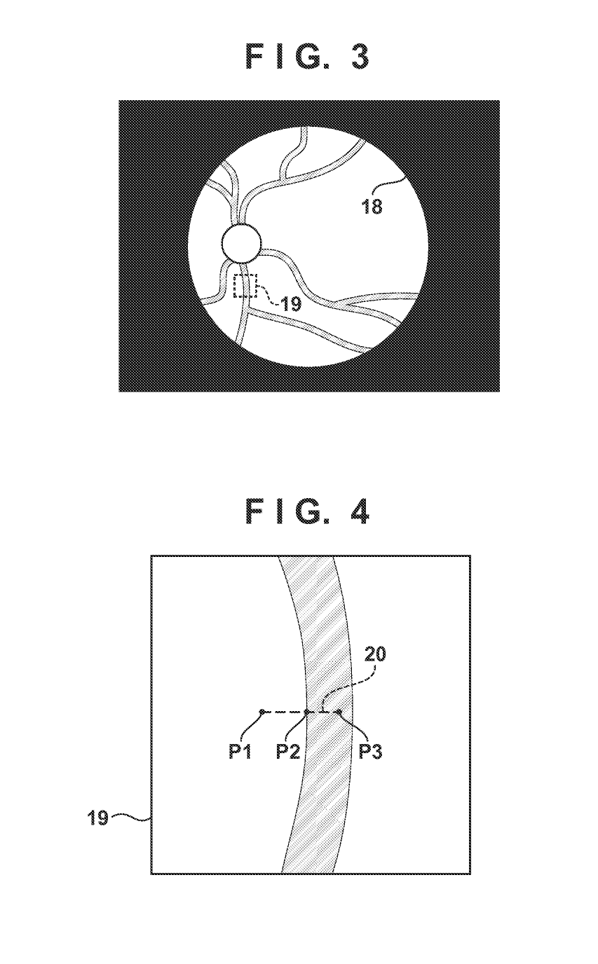

The fundus image autofocus unit 13 then executes fundus image autofocusing (step S106). In fundus image autofocusing, the fundus image autofocus unit 13 performs focusing evaluation by using the object image obtained by imaging the object using the imaging device 5. The fundus image autofocus unit 13 automatically focuses the fundus imaging optical system on the fundus Er based on this focusing evaluation. That is, the fundus image autofocus unit 13 receives the fundus observation image captured by the imaging device 5 whose amplification factor setting has been changed in step S105, while illuminating the fundus Er using the observation light source 10 whose light amount setting has been changed in step S104. The fundus image autofocus unit 13 sets a predetermined area in the received fundus observation image as a focusing evaluation area. In this case, a focusing evaluation area is an area for indicating a specific region of interest in a fundus observation image to which fundus image autofocusing is to be executed. FIG. 3 shows an example of a focusing evaluation area in a fundus observation image. Referring to FIG. 3, a region extending from a portion where a fundus observation image in a mask 18 is depicted to a portion where a large vessel is depicted is set as a focusing evaluation area 19. Note that although the depicted portion of the large vessel is set as the focusing evaluation area 19, another depicted portion such as a papillary region may be set as a focusing evaluation area. The operator may designate a portion at a desired position from a fundus observation image as the focusing evaluation area 19. Alternatively, a predetermined specific region of a fundus observation image may be set as the focusing evaluation area 19.

FIG. 4 shows only the extracted focusing evaluation area 19 set in FIG. 3. The fundus image autofocus unit 13 drives the focus lens 3 to search the set focusing evaluation area 19 for a focus lens position at which the maximum focusing evaluation value is obtained. This focusing evaluation value is the magnitude of the tone difference between structures of the fundus observation image depicted in the focusing evaluation area.

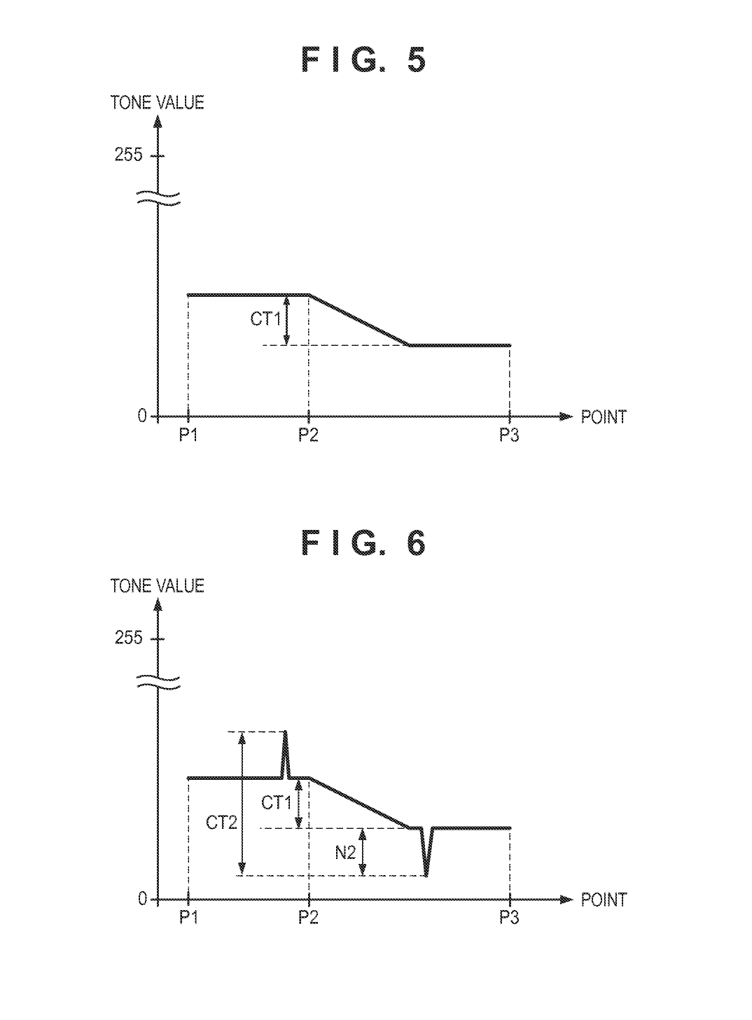

FIG. 5 shows the tone values at points P1 to P3 on a dotted line 20 shown in FIG. 4. Referring to FIGS. 4 and 5, a portion from the point P1 to the point P2 is a depicted portion of the nerve fiber layer, a portion at the point P2 is a depicted portion of the boundary between the blood vessel and the nerve fiber layer, and a portion from the point P2 to the point P3 is a depicted portion of the blood vessel. Noise from the imaging device 5 is superimposed on each tone value in reality. However, for the sake of descriptive convenience, FIG. 5 shows an ideal state in which each tone value is free from the influence of noise generated by the imaging device 5.

In this case, a focusing evaluation value is a tone difference CT1 between a nerve fiber layer portion and a blood vessel portion. The fundus image autofocus unit 13 searches for a focus lens position at which the focusing evaluation value CT1 is maximized, and moves the focus lens to the position after the search, thereby completing the fundus image autofocusing (step S106). Upon completion of the fundus image autofocusing, the SN control unit 15 changes the emitted light amount of the observation light source 10 from I2 to I1 (step S107). The observation light source 10 then emits light at the emitted light amount I1 set by the SN control unit 15. At the same time, the SN control unit 15 changes the amplification factor of the imaging device 5 from S2 to S1 (step S108). The imaging device 5 then captures a fundus observation image with the set amplification factor S1.

An imaging procedure will be described next. Upon completing precise positioning and fundus image autofocusing between the eye E and the non-mydriatic fundus camera 100 described above, the operator can perform imaging by operating an imaging start switch (not shown).

When the operator operates the imaging start switch, the fundus camera control unit 14 causes the imaging light source 12 to emit light. The imaging illumination light emitted by the imaging light source 12 illuminates the fundus Er upon passing through the fundus illumination optical system extending from the imaging light source 12 to the objective lens 1. The reflected light from the fundus Er illuminated by the imaging light source 12 reaches the imaging device 5 through the fundus imaging optical system extending from the objective lens 1 to the imaging lens 4 through the perforated mirror 2 and the focus lens 3. The display image processing unit 16 performs color hue conversion processing and gamma curve computation processing for the fundus image captured by the imaging device 5, and displays the resultant image on the display unit 17.

This embodiment can implement accurate fundus image autofocusing at the operating time of fundus image autofocusing by switching the emitted light amounts of the observation light source 10 and the amplification factors of the imaging device 5 in accordance with whether fundus image autofocusing is active in the above manner. In addition, observation image luminance maintaining processing (to be described above) allows the examiner to focus his/her attention to positioning between the apparatus and the eye to be examined without awareness of autofocusing operation or non-autofocusing operation. These operations will be described separately at the times when fundus image autofocusing is active and when it is inactive.

<Fundus Image Autofocusing: Inactive>

Observation activity to be performed when fundus image autofocusing is inactive is positioning between the eye E and the non-mydriatic fundus camera 100 in the upward, downward, leftward, rightward, forward, and backward directions. The fundus observation image displayed on the display unit 17 is required to allow the operator to recognize the relative positional relationship between the non-mydriatic fundus camera 100 and structures of the fundus such as the papilla, macula, and blood vessel when seeing the overall fundus observation image.

For this reason, the emitted light amount of the observation light source 10 is set to a small value (I1) as much as possible in the required range described above, while the amplification factor of the imaging device 5 is set to a large value (S1) as much as possible in the required range described above. This makes it possible to perform positioning without illuminating an object with an unnecessarily large amount of observation illumination light, that is, without imposing any unnecessarily heavy load on the object.

On the other hand, setting the amplification factor of the imaging device 5 to the large value (S1) will also amplify noise from the imaging device 5, resulting in a decrease in the signal-to-noise ratio between the fundus observation image and noise from the imaging device 5. This makes the operator recognize the noise from the imaging device 5 when precisely observing part of the fundus observation image upon enlarging it. When performing positioning, however, it is only required to recognize the relative positional relationship between the non-mydriatic fundus camera 100 and structures of the fundus such as the papilla, macula, and blood vessel when seeing the overall fundus observation image. Such a low signal-to-noise ratio poses no serious problem.

<Fundus Image Autofocusing: Active>

In fundus image autofocusing, focusing evaluation is performed for a fundus observation image as described in step S106. However, the tone differences between structures of this fundus observation image are very small. For example, the tone difference CT1 between the nerve fiber layer and the blood vessel portion is about 5 to 15. For this reason, noise from the imaging device 5 has a great influence on the focusing accuracy of fundus image autofocusing.

The following is how noise influences the focusing accuracy of fundus image autofocusing. FIG. 6 shows the tone values at the points P1 to P3 (FIG. 4) when the emitted light amount I1 of the observation light source 10 and the amplification factor S1 of the imaging device 5 are set in the same manner as when fundus image autofocusing is inactive. As is obvious, since the amplification factor S1 of the imaging device 5 has a high value, noise N2 from the imaging device 5 is superimposed on the tone value as compared with that in FIG. 5. The higher the amplification factor of the imaging device 5, the larger the magnitude of the noise N2 from the imaging device 5, and vice versa.

When executing fundus image autofocusing in such a case, the apparatus calculates a tone difference CT2 due to the influence of noise N2 in spite of the necessity to calculate the tone difference CT1, resulting in a great reduction in focusing accuracy. For this reason, at the time of fundus image autofocusing operation, the apparatus sets the emitted light amount of the observation light source 10 to a maximum value (I2), and sets the the amplification factor of the imaging device 5 to the small value (S2). That is, the apparatus makes settings to increase the signal-to-noise ratio between a fundus observation image and noise from the imaging device 5. These settings can implement high focusing accuracy.

<Observation Image Luminance Maintaining Processing>

The above described advantage can be obtained by switching the imaging settings in accordance with whether fundus image autofocusing is active, for example, by switching the emitted light amount of the observation light source 10 and the amplification factor of the imaging device 5 according to I2>I1 and S1>S2. However, the fundus image displayed on the display unit 17 changes in luminance at the times when the fundus image autofocusing is active and when it is inactive. This phenomenon may make it difficult for the operator to perform operation due to changes in observed display image. This embodiment therefore performs display control to process the object image obtained by the imaging device 5 so as to maintain the tonality of the image displayed on the display unit 17 before and after changes in imaging settings and display the processed object image on the display unit 17. More specifically, the embodiment performs display control to cause the display image processing unit 16 to perform luminance maintaining processing for a captured image and then cause the display unit 17 to display the resultant image as an observation image. This arrangement performs display upon correcting changes in tone caused by changes at the times when fundus image autofocusing is active and when it is inactive, and maintains the tonality of the display image at the times when fundus image autofocusing is active and when it is inactive. This can implement an environment in which the examiner can perform operation without awareness of autofocusing operation.

A method of adjusting a gamma curve used for display processing for the display unit 17 will be described below as an example of observation luminance maintaining processing in this embodiment.

For example, the tone value of a fundus observation image may be maintained by satisfying the following equation:

α×I1×S1×γ1=α×I2×S2×γ2 (1)

In this case, the left-hand side “α×I1×S1×γ1” represents the tone value of the fundus observation image displayed on the display unit 17 when fundus image autofocusing is inactive, and the right-hand side “α×I2×S2×γ2” represents the tone value of the fundus observation image displayed on the display unit 17 when fundus image autofocusing is active. In addition, α represents the spectral reflectance coefficient of the fundus, γ1 represents a γ curve processed by the display image processing unit 16 when fundus image autofocusing is inactive, and γ2 represents a y curve processed by the display image processing unit 16 when fundus image autofocusing is active. Obviously, it is also possible to adjust a gamma curve by using I1, I2, S1, and S2 within the range in which equation (1) is satisfied.

Therefore, the gamma curve γ2 for maintaining the tone value set when fundus image autofocusing is inactive during the time when fundus image autofocusing is active is calculated according to

γ2=γ1×(I1×S1)/(I2×S2) (2)

The display image processing unit 16 displays an image having this gamma curve value on the display unit 17 when fundus image autofocusing is active.

Note that since the display unit 17 displays a fundus observation image even when fundus image autofocusing is active, the operator can perform positioning between the eye E and the non-mydriatic fundus camera 100 in the upward, downward, leftward, rightward, forward, and backward directions.

As described above, according to this embodiment, it is possible to maintain the luminance of an observation image constant even if the emitted light amount setting of the observation light source 10 and the amplification factor setting of the imaging device 5 are changed to increase the S/N ratio when fundus image autofocusing is active. This allows the operator to observe the fundus image displayed on the display unit 17 without any feeling of strangeness even when fundus image autofocusing is active.

In the above embodiment, in order to increase the signal-to-noise ratio between a fundus observation image and noise from the imaging device 5 at the time when fundus image autofocusing is active as compared with the time when fundus image autofocusing is inactive, the apparatus sets the emitted light amount of the observation light source 10 to I2 and the amplification factor of the imaging device 5 to S2. However, the present invention is not limited to this. The apparatus can increase the signal-to-noise ratio between a fundus observation image and noise from the imaging device 5 even by setting the emitted light amount of the observation light source 10 to I2 while keeping the amplification factor of the imaging device 5 at S1. Likewise, the apparatus can also increase the signal-to-noise ratio between a fundus observation image and noise from the imaging device 5 by keeping the emitted light amount of the observation light source 10 at I1 while changing the amplification factor of the imaging device 5 from S1 to S2. In either case, it is possible to maintain the luminance of the image displayed on the display unit 17 constant by executing observation image luminance maintaining processing described above.

The above embodiment changes the amplification factor setting of the imaging device 5 to increase the signal-to-noise ratio between a fundus observation image and noise from the imaging device 5 at the time when fundus image autofocusing is active as compared with the time when fundus image autofocusing is inactive. However, it is also possible to increase the signal-to-noise ratio between a fundus observation image and noise from the imaging device 5 by changing the charge accumulation period of the imaging device 5. For example, letting SP1 be the charge accumulation period when fundus image autofocusing is inactive, and SP2 be the charge accumulation period when fundus image autofocusing is active, the apparatus may perform operation so as to satisfy SP1>SP2. In this case, substituting SP1 and SP2 for S1 and S2 in equation (2) can implement observation luminance maintaining processing.

Although the above embodiment has exemplified the case of performing fundus image autofocusing, it is possible to obtain the same effect as that described above even when the operator manually performs focusing without performing fundus image autofocusing while seeing the fundus observation image captured by the imaging device 5. More specifically, when the apparatus shifts to the manual focusing mode in which the apparatus moves the focus lens 3 in accordance with an operation input from the operator to achieve an in-focus state, the imaging control apparatus determines that the focus operation started and changes the imaging settings to those for improving the signal-to-noise ratio of the image obtained by imaging. Alternatively, the apparatus may be provided with a detection unit which detects that the focus lens 3 is manually operated, and may determine from the detection result obtained by the detection unit whether focusing operation is active or inactive. When focusing operation is inactive, the apparatus sets the signal-to-noise ratio between a fundus observation image and noise from the imaging device 5 to a lower setting to reduce the load on the object. When focusing operation is active, the apparatus sets the the signal-to-noise ratio between a fundus observation image and noise from the imaging device 5 to a higher setting than that when focusing operation is inactive. This makes it possible to provide an accurate in-focus state even at the time of manual focusing operation. In addition, at this time, since the apparatus executes observation luminance maintaining processing, the operator can see images with uniform luminance at both the times when the focus lens 3 is operated and when it is not operated.

Aspects of the present invention can also be realized by a computer of a system or apparatus (or devices such as a CPU or MPU) that reads out and executes a program recorded on a memory device to perform the functions of the above-described embodiment(s), and by a method, the steps of which are performed by a computer of a system or apparatus by, for example, reading out and executing a program recorded on a memory device to perform the functions of the above-described embodiment(s). For this purpose, the program is provided to the computer for example via a network or from a recording medium of various types serving as the memory device (for example, computer-readable storage medium).

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-242214, filed Nov. 1, 2012, which is hereby incorporated by reference herein in its entirety.

What is claimed is:

1. An imaging control apparatus comprising:
a focusing unit configured to set an in-focus state of an imaging optical system on an object by using an object image obtained by imaging the object illuminated by a light source by using an imaging device;
a change unit configured to change an imaging setting to change a signal-to-noise ratio of the object image obtained by the imaging at the time of executing focusing operation using said focusing unit and at other times; and
a display control unit configured to process the object image so as to maintain tonality of the image displayed on a display unit before and after said change unit changes the imaging setting and display the processed object image on said display unit.

2. The apparatus according to claim 1, wherein said change unit increases an emitted light amount of the light source during execution of the focusing operation relative to the emitted light amount during nonexecution of the focusing operation.

3. The apparatus according to claim 1, wherein said change unit decreases an amplification factor of a signal from the imaging device during executing of the focusing operation relative to the amplification factor during nonexecution of the focusing operation.

4. The apparatus according to claim 1, wherein said change unit shortens a charge accumulation period in the imaging device during execution of the focusing operation relative to the charge accumulation period during nonexecution of the focusing operation.

5. The apparatus according to claim 1, wherein said focusing unit performs focusing evaluation by using an object image obtained by the imaging, and automatically sets the imaging optical system in the in-focus state based on the focusing evaluation.

6. The apparatus according to claim 1, wherein in the focusing operation by said focusing unit, a focus lens is moved in the imaging optical system in accordance with an operation input from an operator.

7. The apparatus according to claim 1, wherein said display control unit processes the object image by changing a gamma curve used for display processing so as to maintain the tonality before and after said change unit changes the imaging setting.

8. An ophthalmic apparatus comprising:
an imaging control apparatus defined in claim 1;
a light source;
an imaging device; and
an imaging optical system,
wherein a fundus is imaged as an object.

9. An imaging control method comprising:
a step of setting an in-focus state of an imaging optical system on an object by using an object image obtained by imaging an object illuminated by a light source by using an imaging device;
a step of changing an imaging setting to change a signal-to-noise ratio of the object image obtained by the imaging at the time of executing focusing operation in the step of setting the in-focus state and at other times; and
a step of processing the object image so as to maintain tonality of the image displayed on display unit before and after the imaging setting is changed in the step of changing and displaying the processed object image on the display unit.

10. A non-transitory computer readable storage medium storing a program for causing a computer to execute each step in an imaging control method defined in claim 9.
